(19) United States
(12) Patent Application Publication
(10) Pub. No.: US 2009/0263286 A1
(43) Pub. Date: Oct. 22, 2009

(54) SENSOR

(76) Inventors: Hiroshi ISOMURA, Nagoya-shi (JP); Takaya Yoshikawa, Kasugai-shi (JP)

Correspondence Address: STITES & HARBISON PLLC, 1199 NORTH FAIRFAX STREET, SUITE 900, ALEXANDRIA, VA, 22314, US

(21) Appl. No.: 12/424,985

(22) Filed: Apr. 16, 2009

(30) Foreign Application Priority Data
Apr. 16, 2008 (JP) P2008-106921
Jan. 28, 2009 (JP) P2009-016281

(51) Int. Cl. G01N 33/00

(52) U.S. Cl. 422/83

(57) ABSTRACT

A sensor includes: a sensor element extending in an axial direction comprising a leading end portion exposed to a gas measurement object; a metal shell covering a periphery of the sensor element; an outer cylinder member fixed to a rear end side of the metal shell, the outer cylinder member being composed of a first metal material; and a protection outer cylinder member covering an outer surface of the outer cylinder member, the protection outer cylinder being composed of a second metal material. The outer cylinder member further includes a sealing part in contact with a circumference of the protection outer cylinder member, and a spaced part positioned closer to a leading end side than the sealing part, and spaced from the protection outer cylinder member. The first metal material is less subject to rusting than the second metal material.

### CROSS REFERENCE TO RELATED APPLICATION

The present application claims priority to Japanese Patent Application No. 2008-106921 filed on Apr. 16, 2008, and to Japanese Patent Application No. 2009-016281 filed on Jan. 28, 2009, the disclosures of which are incorporated herein by reference in their entirety.

### BACKGROUND OF THE INVENTION

1. Field of the Invention

The present invention relates to sensors, such as gas sensors (e.g., oxygen sensors, hydrocarbon sensors, nitrogen oxide sensors) for detecting or measuring a gas measurement object contained in an atmosphere, and temperature sensors for measuring a temperature of the atmosphere.

2. Description of Related Art

Such sensors generally have a plurality of cylindrical components, including, for example: a sensor element having a detection part disposed at a leading end thereof for detecting or measuring the measurement object; a metal shell having an opening part at a rear end side thereof, a leading end, and a thread part formed on an outer periphery thereof, where the sensor extends from the opening part and the detection part of the sensor element projects from the leading end; a protector element fixed to the metal shell in such a fashion as to cover the detection part of the sensor element; an outer cylinder member having a periphery and being fixed to the opening part of the metal shell for protecting the sensor element; a protection outer cylinder member covering the periphery of the outer cylinder member and being disposed via a water repellent filter; and the like. The thread part formed on the outer periphery of the metal shell is for attaching the sensor to a structure, such as an exhaust pipe.

For example, an oxygen sensor is generally mounted to an exhaust pipe for at a downstream side of a catalyst device. The catalyst device is for decomposing an organic substance in an exhaust gas. The sensor measures a detection object component contained in the exhaust gas from which the organic substance is decomposed or eliminated. The exhaust pipe extends along a bottom part of an vehicle. Therefore, water that is splashed during travel of the vehicle may adhere to an outer surface of the gas sensor as water droplets. In order to prevent moisture from entering the inside of the gas sensor, it is necessary to ensure sufficient water tightness of the gas sensor by reliably joining the plurality of cylindrical bodies.

As a method for joining the plurality of cylindrical bodies, a caulk-fixing method comprises the following steps: the protector is caulk-fixed to the leading end of the metal shell; a leading end of the outer cylinder member is caulk-fixed to the rear end part of the metal shell; and a leading end of the protection outer cylinder member is caulk-fixed to the outer cylinder member (See: Patent Publication JP-A-11-352095). Further, a caulking part is provided at the leading end side of the protection outer cylinder member in such a fashion as to be reduced in diameter to tightly contact an outer surface of the outer cylinder member to prevent entrance of moisture and the like between the outer cylinder member and the protection outer cylinder member.

FIG. 5 shows a typical configuration of a leading end of a protection outer cylinder member 12 caulk-fixed to an outer cylinder member 11 at a position spaced from the leading end of the protection outer cylinder member 12. This configuration allows a positional shift of the components within an error range. However, a remaining part of the protection outer cylinder member 12 that is forward of a caulking part S4 is warped outwardly, and a very small gap is often formed between the leading end of the protection outer cylinder member 12 and the outer cylinder member 11. In the case where such gap is formed, water droplets adhered to a periphery of the outer surface of the gas sensor are drawn to the gap by capillary action, and the moisture drawn to the gap is retained in the gap for a long time by surface tension.

Particularly, when the “water” is a “saline water” (an aqueous solution containing a metal salt), a chemical reaction occurs between an outer surface of a portion of the outer cylinder member 11 adjacent to the gap (hereinafter, a portion of the outer cylinder member 11 adjacent to the gap is referred to as a spaced part R1) and an inner surface of the protection outer cylinder member 12, increasing the risk of corrosion of both of the outer surface of the spaced part R1 and the inner surface of the protection outer cylinder member 12. For instance, a snow melting agent containing calcium chloride as a main component is generally used in snowy cold districts, creating saline water puddles on the ground. When a vehicle drives through the puddles, splashed saline water adheres to the gas sensor and is drawn into the gap, thereby raising the risk of corrosion. Particularly, when the corrosion progresses to the spaced part R1, a crack C can form in the spaced part R1, allowing the saline water to enter the inside of the outer cylinder member 11 from the crack C. Ingress of saline water into the outer cylinder member 11 can cause a decrease (i.e., raising the risk of a reduction in) the detection or measurement accuracy of the sensor.

### BRIEF SUMMARY OF THE INVENTION

The present invention solves the above-described disadvantages, and an object thereof is to provide a sensor having a structure in which a plurality of cylindrical bodies are combined to one another, wherein a corrosion of an outer cylinder member otherwise caused by adhesion of moisture such as saline water is suppressed, and a reduction in detection or measurement accuracy of the sensor otherwise caused by entrance of the saline water or the like into the inside of the outer cylinder member is prevented.

According to a first aspect of the invention, a sensor includes: a sensor element extending in an axial direction, having a periphery and comprising a leading end portion exposed to a gas measurement object; a metal shell covering the periphery of the sensor element and comprising a rear end side; an outer cylinder member fixed to the rear end side of the metal shell, the outer cylinder member being composed of a first metal material, having an outer surface and comprising a leading end side; and a protection outer cylinder member covering the outer surface of the outer cylinder member, the protection outer cylinder being composed of a second metal material and having a circumference. The outer cylinder member further includes a sealing part in contact with the circumference of the protection outer cylinder member, and a spaced part positioned closer to the leading end side than the sealing part, opposed to the protection outer cylinder member, and spaced from the protection outer cylinder member. The first metal material is less subject to rusting than the second metal material.

Advantageously, since the first metal material is less subject to rusting than the second metal material, the protection outer cylinder member is corroded before the outer cylinder member even when saline water or the like is retained in the gap between an inner peripheral surface of the protection outer cylinder member and an outer peripheral surface of the spaced part, thereby making it possible to suppress corrosion of the outer cylinder member. As a result, a crack C in the spaced part is suppressed, and the saline water is suppressed from entering the inside of the outer cylinder member. Therefore, a reduction in detection accuracy of the sensor is also suppressed.

In accordance with one implementation, the first metal material and the second metal material are stainless steels, and the first metal material has a larger Cr content (wt %) than the second metal material. Further, the first metal material may have a larger Ni content (wt %) than the second metal material. Further still, the first metal material may be SUS310S, and the second metal material may be SUS304L.

In accordance with another implementation, the sealing part is in contact with the protection outer cylinder member directly. Alternately, the sealing part may be in contact with the protection outer cylinder member indirectly via another member.

Other features and advantages of the invention will be set forth in or apparent from the detailed description of the exemplary embodiments of the invention found below.

### BRIEF DESCRIPTION OF THE DRAWINGS

Illustrative aspects of the invention will be described in detail with reference to the following figures.

FIG. 1 is a sectional view of a gas sensor according to a first exemplary embodiment of the invention.

FIG. 2 is an enlarged view of a portion of the gas sensor of FIG. 1.

FIG. 3 is a sectional view of a gas sensor according to a second exemplary embodiment of the invention.

FIG. 4 is an enlarged view of a portion of the gas sensor of FIG. 3.

FIG. 5 is a partial sectional view of an exemplary gas sensor showing a crack C formed in an outer cylinder member combined with a protection outer cylinder member in a related example.

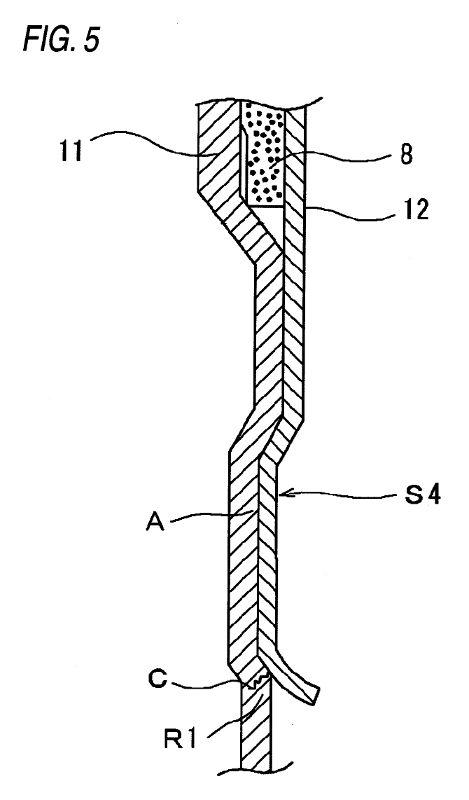

### DETAILED DESCRIPTION OF EXEMPLARY EMBODIMENTS OF THE INVENTION

Hereinafter, an first exemplary gas sensor 100 and a second exemplary gas sensor 200 according to the invention will be described with reference to the drawings.

1. Gas Sensor according to First Exemplary Embodiment

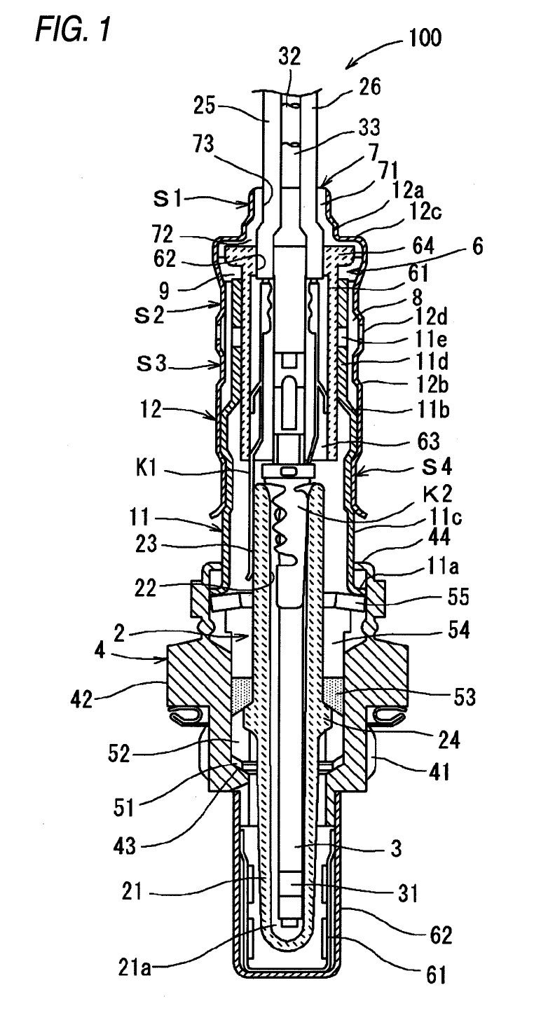

FIG. 1 is a sectional view schematically showing the gas sensor 100 according to a first exemplary embodiment of the invention. In use, the gas sensor 100 is attached to an exhaust pipe of an automobile. An example of the gas sensor 100 is an oxygen sensor for detecting a concentration of oxygen contained in an exhaust gas flowing inside an exhaust pipe. The oxygen sensor will hereinafter be described in detail.

The gas sensor 100 extends in an axial direction and is provided with a sensor element 2 in the form of a bottomed cylinder having a closed leading end part. A metal shell 4 covers a periphery of the sensor element 2 and retains the sensor element 2 at its inner surface side. An outer cylinder member 11 is fixed to a rear end side of the metal shell 4 and is in the form of a cylinder. A protection outer cylinder member 12 covers an outer periphery of the outer cylinder member 11 and is in the form of a cylinder.

In this embodiment, in the axial direction of the sensor 100 of FIG. 1, a part in a direction of a leading end part that is exposed to a gas measurement object (exhaust gas) of the sensor element 2 (close to a bottom part of a solid electrolyte body 21 in the form of a bottomed cylinder, i.e. a lower part of FIG. 1) is referred to as a “leading end side”, and a part in a reverse direction (upper part of FIG. 1) is referred to as a “rear end side”.

The sensor element 2 is obtained by molding using a ceramic containing as a main component partially stabilized zirconium or the like obtained by dissolving yttrium or the like as a stabilizer and having oxygen ion conductivity. The sensor element 2 has the solid electrolyte body 21 in the form of a bottomed cylinder with a closed leading end part, a porous inner electrode layer 22 formed on substantially an entire inner surface of the solid electrolyte body 21 and made from Pt or a Pt alloy, and a porous outer electrode layer 23 formed in the same manner as the inner electrode layer 22 on an outer surface of the solid electrolyte body 21. Also, the outer electrode layer 23 is covered with a porous electrode protection layer (not shown) made from a heat resistant ceramic such as alumina magnesia spinel or the like. Further, an engagement flange part 24 projected in a radially outward direction is provided at substantially the middle position in the axial direction of the sensor element 2.

A ceramic heater 3 having a heating part 31 and being in the form of a stick is inserted into the solid electrolyte body 21 in such a fashion that the heating part 31 is positioned at a bottom part side of the solid electrolyte body 21. In the ceramic heater 3, the heating part 31 generates heat when energized via heater lead wires 32 and 33 described later in this specification, and the sensor 2 is heated to be activated.

The metal shell 4 has a thread part 41 for attaching the gas sensor 100 to an attachment part of the exhaust pipe and a hexagonal part 42 to which a mounting tool is abutted for the attachment to the exhaust pipe. A metal fitting step part 43 projecting in the radially inward direction is provided on an inner periphery at a leading end side of the metal shell 4, and a support member 52 made from alumina is latched via a packing 51 on the meal fitting step part 43. The sensor element 2 is supported by the metal shell 4 when an engagement flange part 24 is supported by the support member 52. Further, a filler part 53 in which an inorganic filler such as a talc powder is filled is formed between an inner surface of the metal shell 4 and an outer surface of the sensor element 2 at a rear end side of the support member 52, and a sleeve 54 made from alumina and an annular ring 55 are sequentially and coaxially inserted into a rear end side of the filler member 53.

Double protectors 61 and 62 made from a metal are fixed by welding to an outer periphery at the leading end side of the metal shell 4 in such a fashion as to enclose the leading end part of the sensor element 2 projecting from a leading end of the metal shell 4. A plurality of gas inlet holes are formed on each of the protectors 61 and 62, and an exhaust gas flows in from the gas inlet holes, so that the concentration of oxygen contained in the exhaust gas is detected.

The outer cylinder member 11 is composed of (or formed by using) SUS310S (i.e., a first metal material) having a thickness of 0.8 mm, and the leading end side of the outer cylinder 11 is inserted into the inside at the rear end side of the metal shell 4 to be fixed. The outer cylinder member 11 is fixed to the metal shell 4 by caulking of a metal fitting rear end part 44 of the metal shell 4 in a state where a leading end opening end part 11a that is an opening end part having an enlarged diameter at the leading end side is abutted to the annular ring 55.

The gas sensor 100 has a structure that the filler part 53 is formed when the inorganic filler such as a talc powder is compressed and filled via the sleeve 54 when the metal fitting rear end part 44 of the metal shell 4 is caulked. With such structure, the sensor element 2 is retained inside the cylindrical metal shell 4 in a water-tight state.

Further, a step part 11b is formed on the outer cylinder member 11 at substantially the middle position in the axial direction, wherein the leading end side from the step part 11b is formed as an leading end side part 11c, and a rear end side from the step part 11b is formed as an rear end side part 11d. The rear end side part 11d has an inner diameter and an outer diameter that are slightly smaller than those of the leading end side part 11c, and the inner diameter is slightly larger than an outer diameter of a separator main body part 61 of a separator 6 described later in this specification. Also, a plurality of air inlet holes 11e are formed on the rear end side part 11d along a circumference and at a predetermined interval.

The protection outer cylinder member 12 is composed of a second metal material, and is molded in the form of a cylinder by deep-drawing a plate material of SUS304L having a thickness of 0.4 mm. The protection outer cylinder member 12 has a rear end side part 12a having an opening part in communication with the inside from the outside and formed on the rear end side, a leading end side part 12b coaxially fitted from the rear end side into the outer cylinder member 11 and formed on the leading end side, and a modified diameter part 12c formed between the rear end side part 12a and the leading end side part 12b. Also, a plurality of air inlet holes 12d are formed on the leading end side part 12b of the protection outer cylinder member 12 along a circumference and at a predetermined interval.

A caulking part S1 is formed on the rear end side part 12a for fixing an elastic sealing member 7 described later in this specification in a water tight state.

A filter 8 is disposed at a position corresponding to the air inlet holes 12d and 11e between the protection outer cylinder member 12 and the outer cylinder member 11. It is possible to prevent moisture from entering from the air inlet holes 11e by the filter 8. The filter 8 is formed from a porous body made from a fiber made from a synthetic resin and the like and may particularly preferably be a filter formed from a porous body made from a fiber excellent in water repellant property. Examples of such filter 8 include a porous body made from a polytetrafluoroethylene fiber (manufactured by Japan Gore-Tex, trade name: Gore-Tex) and the like, and the filter 8 is capable of suppressing permeation of water or a liquid containing a large amount of water as well as of readily allowing permeation of a gas such as the air.

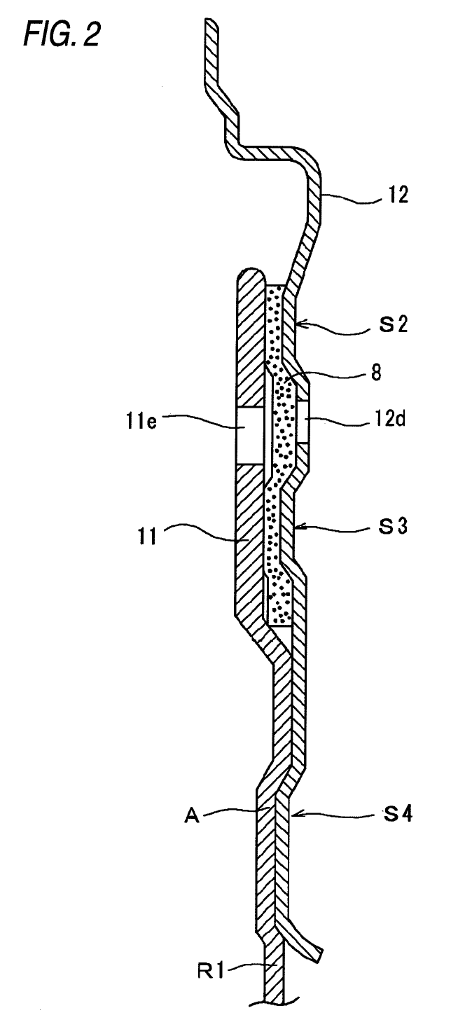

As shown in FIG. 2, the protection outer cylinder member 12 and the outer cylinder member 11 are fixed by a second caulking part S2 formed by caulking at least a part at the rear end side from the air inlet hole 12d in the radially inward direction via the filter 8 and a third caulking part S3 formed by caulking at least a part at the leading end side from the air inlet hole 12d in the radially inward direction via the filter 8. In this case, the filter 8 is retained as being compressed between the outer cylinder member 11 and the protection outer cylinder member 12. The leading end side part 12b of the protection outer cylinder member 12 is disposed in such a fashion as to be fitted into the outer cylinder leading end side barrel part 11c from the outside. Further, a caulking part S4 that is reduced in diameter in the radially inward direction is formed by caulking the leading end part of the protection outer cylinder leading end part 12b and the outer cylinder leading end side barrel part 11c, i.e. by caulking the leading end part of the protection outer cylinder leading end side part 12b in the radially inward direction.

The outer cylinder member 11 and the protection outer cylinder member 12 are firmly fixed to each other by caulk-fixing the protection outer cylinder member 12 to the outer cylinder member 11 as described above. The air serving as a reference gas is introduced into the inside of the outer cylinder member 11 through the air inlet hole 12d, the filter 8, and the air inlet holes 11e to be introduced to the bottom part 21a of the solid electrolyte body 21. The moisture is suppressed from flowing by the filter 8 to be inhibited from entering the inside of the outer cylinder member 11.

Also, it is possible to combine and fix the outer cylinder member 11 and the protection outer cylinder member 12 not only by the caulking but also by a method such as welding such as resistance welding, laser beam welding, and electron beam welding and press fitting.

The outer cylinder member 11 and the protection outer cylinder member 12 are caulked at the caulking part S4 to be combined and fixed to each other. In the combining and fixing, the outer cylinder member 11 has a sealing part A (part corresponding to the caulking part S4) contacting the protection outer cylinder member 12 along a circumference and a spaced part R1 spaced from and opposed to the protection outer cylinder member 12. Also, the sealing part A and the spaced part R1 are likewise formed in the case of welding and press fitting.

Even when moisture enters in a gap between the inner peripheral surface of the leading end part of the protection outer cylinder member 12 and the outer peripheral surface of the spaced part R1 of the outer cylinder member 11 and is retained, the protection outer cylinder member 12 is corroded before the outer cylinder member 11 when the outer cylinder member 11 is made from SUS310S and the protection outer cylinder member 12 is made from SUS304L. This is because the outer cylinder member 11 is formed by using a material less subject to rusting than the material used for the protection outer cylinder member 12. Therefore, it is possible to suppress corrosion of the outer cylinder member 11, thereby making it possible to suppress a reduction in detection accuracy of the sensor.

As shown in FIG. 1, a substantially cylindrical separator 6 is disposed inside the rear end side part 11d of the outer cylinder member 11. A lead wire insertion hole 62 to which element lead wires 25 and 26 and the heater lead wires 32 and 33 are inserted is formed on the separator 6 in such a fashion as to penetrate through from the leading end side to the rear end side. Also, a bottomed retention hole 63 opened at a leading end surface of the separator 6 is formed on the separator 6 in the axial direction. A rear end part of the ceramic heater 3 is inserted into the inside of the retention hole 63, and positioning of the ceramic heater 3 in the axial direction is established when a rear end surface of the ceramic heater 3 contacts a bottom surface of the retention hole 63.

Further, the separator 6 is provided with a separator main body part 61 fitted into the inside at the rear end side of the outer cylinder member 11 and a separator flange part 64 extending in a radially outward direction from a rear end part of the separator main body part 61. That is, the separator 6 is disposed inside the protection outer cylinder member 12 in a state where the separator main body part 61 is fitted into the outer cylinder member 11 and the separator flange part 64 is supported by a rear end surface of the outer cylinder member 11 via an annular sealing member 9 made from a fluorine rubber or the like.

An elastic sealing member 7, excellent in heat resistance and made from a fluorine rubber or the like, is disposed at the rear end side of the separator 6. The elastic sealing member 7 has a main body part 71 and a sealing member guard part 72 extending in the radially outward direction at the leading end side of the main body part 71. Also, four lead wire insertion holes 73 are formed in such a fashion as to penetrate though the main body part 71 in the axial direction. As described above, the elastic sealing member 7 is inserted into the inside at the rear end side of the protection outer cylinder member 12, and the protection outer cylinder member 12 is caulked to form the caulking part S1, thereby fixing the elastic sealing member 7 to the protection outer cylinder member 12.

Further, the element lead wires 25 and 26 and the heater lead wires 32 and 33 are inserted into the separator lead wire insertion hole 62 of the separator 6 and the lead wire insertion hole 73 of the elastic sealing member 7 to be drawn to the outside from the inside of the outer cylinder member 11 and the protection outer cylinder member 12.

The four lead wires 32, 33, 25, and 26 are connected to a connector (not shown) at the outside, and input and output of electric signals between external appliances such as an ECU and the lead wires 32, 33, 25, and 26 are performed via the connector.

The lead wires 32, 33, 25, and 26 have a structure that a conductor is coated with an insulating film made from a resin, and a rear end part of the conductor is connected to a connecter terminal provided in the connector (not shown in detail). A leading end part of the conductor of the element lead wire 25 is caulked at the rear end part of the terminal metal fitting K1 externally mounted on an outer surface of the solid electrolyte body 21, and a leading end part of the conductor of the element lead wire 26 is caulked at a rear end part of a terminal metal fitting K2 that is press-fitted into the inside of the solid electrolyte body 21 for the connection. With such constitution, the element lead wire 25 is electrically connected to the outer electrode layer 23 of the sensor element 2, and the element lead wire 26 is electrically connected to the inner electrode layer 22. Leading end parts of the conductors of the heater lead wires 32 and 33 are connected to a pair of heater terminal metal fittings joined to a heating resistor of the ceramic heater 3.

2. Gas Sensor according to Second Exemplary Embodiment

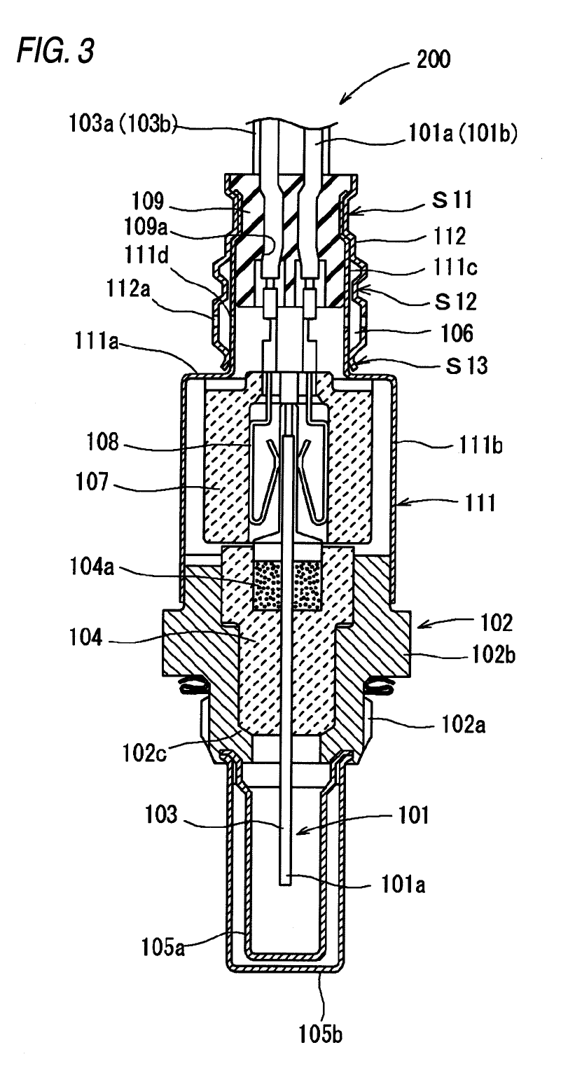

Without limitation to the gas sensor 100 according to the above-described first exemplary embodiment, the same effect is achieved by the gas sensor 200 according to the second exemplary embodiment as shown in FIG. 3.

The gas sensor 200 is provided with a plate-like sensor element 101 extending in an axial direction, a metal shell 102 retaining the sensor element 101 in its inside via another member, an outer cylinder member 111 fixed to a rear end side of the metal shell 102, and a protection outer cylinder member 112 fitted and fixed to an outer periphery of the other end side of the outer cylinder member 111.

In the sensor element 101, a detection part 101a for detecting or measuring a measurement object component contained in an atmosphere and a ceramic heater 103 are formed in an integrated fashion and have the same structure as a related art. The metal shell 102 has a thread part 102a for attaching the gas sensor 200 to an attachment part of the exhaust pipe and a hexagonal part 102b to which a mounting tool is attached for the attachment to the exhaust pipe. A support member 104 made from alumina is latched on a meal fitting step part 102c of the metal shell 102. The sensor element 101 is fixed to the support member 104 by a sealing member 104a made from a glass. Double protectors 105a and 105b made from a metal are fixed by welding at a leading end side of the metal shell 102 in such a fashion as to enclose a leading end part of the sensor element 101 projecting from a leading end of the metal shell 102. A plurality of gas inlet holes are formed on each of the protectors 105a and 105b, and an exhaust gas flows in from the gas inlet holes, so that the concentration of oxygen contained in the exhaust gas is detected.

A leading end side of the outer cylinder member 111 is fitted and fixed to a rear end side of the metal shell 102. The outer cylinder member 111 has a step part 111a formed at substantially the middle position in the axial direction, wherein a part close to the leading end side from the outer cylinder step part 111a is formed as an leading end side part 111b, and a part close to the rear end side from the outer cylinder step part 111a is formed as an rear end side part 111c. Further, a plurality of air inlet holes 111d are formed on the rear end side part 111c along a circumference and at a predetermined interval. A leading end side of the protection outer cylinder member 112 is coaxially fitted into and combined with the rear end side part 111c of the outer cylinder member 111. A plurality of air inlet holes 112a are formed on the protection outer cylinder part 112 along a circumference and at a predetermined interval.

The outer cylinder member 111 is composed of (or formed from) SUS310S (i.e., a first metal material). The protection outer cylinder member 112 is composed of (or formed from) SUS304L (i.e., a second metal material).

A filter 106 is disposed at least at a position corresponding to the air inlet holes 112d and 111d between the protection outer cylinder member 112 and the outer cylinder member 111.

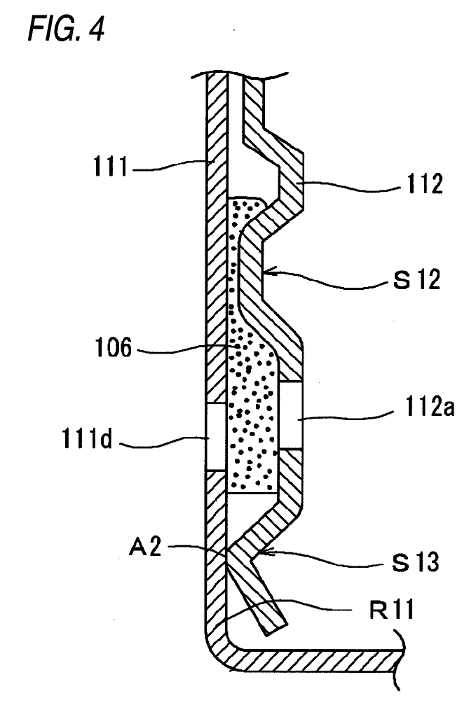

The protection outer cylinder member 112 is caulked in a radially inward direction to be fixed to and combined with the outer cylinder member 111. More specifically, the protection outer cylinder member 112 has a first caulking part S11 by which the protection outer cylinder member 112 and the outer cylinder member 111 are directly caulked at the rear end side of the position where the filter 106 is disposed, a second caulking part S12 that is caulked at the rear end side of the air inlet holes 112a and 111d via the filter 106, and a third caulking part S13 that is caulked at the leading end side of the air inlet holes 112a and 111d (see FIG. 4).

The outer cylinder member 111 and the protection outer cylinder member 112 are caulked at the combining and caulking part S13 to be combined and fixed to each other. In the combining and fixing, the outer cylinder member 111 has a sealing part A2 (part corresponding to the combining and caulking part S13) contacting the protection outer cylinder member 112 at a circumference thereof and a spaced part R11 spaced from and opposed to the protection outer cylinder member 112. Also, the sealing part A2 and the spaced part R11 are likewise formed in the case of welding and press fitting.

Even when moisture enters a gap between the inner peripheral surface of the leading end part of the protection outer cylinder member 112 and the spaced part R1 of the outer peripheral surface of the outer cylinder member 111 and is retained, the protection outer cylinder member 112 is corroded before the outer cylinder member 111 when the outer cylinder member 111 is made from SUS310S and the protection outer cylinder member 112 is made from SUS304L. This is because the outer cylinder member 111 is formed by using a material less subject to rusting than the material used for the protection outer cylinder member 112. Therefore, it is possible to suppress corrosion of the outer cylinder member 111, thereby making it possible to suppress a reduction in detection accuracy of the sensor.

A substantially cylindrical separator 107 is disposed inside the outer cylinder rear end side barrel part 111b of the outer cylinder member 111. Connection terminals 108 (only two connection terminals are shown in FIG. 9) to be connected to element lead wires 101a and 101b and heater lead wires 103a and 103b are inserted into the separator 107. An elastic sealing member 109, which is excellent in heat resistance and made from a fluorine rubber or the like, is disposed inside the outer cylinder rear end side barrel part 111c of the outer cylinder member 111 and four lead wire insertion holes 109a are formed on the elastic sealing member 109 in such a fashion as to penetrate through in the axial direction.

The present invention is useful for various sensors, such as gas sensors (e.g., oxygen sensors, hydrocarbon sensors, and nitrogen oxide sensors) for detecting or measuring a gas measurement object contained in an atmosphere, and temperature sensors for measuring a temperature of the atmosphere.

According to the above, a sensor of an first aspect of the present invention comprises: a sensor element extending in an axial direction, having a periphery and comprising a leading end portion exposed to a gas measurement object; a metal shell covering the periphery of the sensor element and comprising a rear end side; an outer cylinder member fixed to the rear end side of the metal shell, the outer cylinder member being composed of a first metal material, having an outer surface and comprising a leading end side; and a protection outer cylinder member covering the outer surface of the outer cylinder member, the protection outer cylinder being composed of a second metal material and having a circumference. The outer cylinder member further comprises a sealing part in contact with the circumference of the protection outer cylinder member, and a spaced part positioned closer to the leading end side than the sealing part, opposed to the protection outer cylinder member, and spaced from the protection outer cylinder member. The first metal material is less subject to rusting than the second metal material

Also, according to an implementation of the present invention, the first metal material and the second metal material are stainless steels, and the first metal material has a larger Cr content (wt %) than the second metal material.

Also, according to another implementation of the present invention, the first metal material has a larger Ni content (wt %) than the second metal material.

Further, according to a yet another implementation of the present invention, the first metal material is SUS310S, and the second metal material is SUS304L.

According to other implementations of the present invention, the sealing part is in contact with the protection outer cylinder member either directly or indirectly via another member.

According to the sensor of this invention, since the first material (i.e., the material less subject to rusting than the material for the protection outer cylinder member) is used for the outer cylinder member, the material of the protection outer cylinder member (i.e., the second material) is corroded before the outer cylinder member even when saline water or the like is retained in the gap between the inner peripheral surface of the protection outer cylinder member and the outer peripheral surface of the outer cylinder member (spaced part), thereby making it possible to suppress corrosion of the outer cylinder member. As a result, the crack C in the spaced part is suppressed, and the saline water is suppressed from entering the inside of the outer cylinder member, thereby suppressing a reduction in detection accuracy of the sensor.

It is possible to put the wording “The first metal material is less subject to rusting than the second metal material” into practice by the following method. A first test piece made from a predetermined material and a second test piece made from a material different from that of the first test piece are prepared, and the first test piece and the second test piece are tested by a saline water spray test method of JIS Z2371 (version 2007). After the test, weights of the first test piece and the second test piece are measured, and when the weight of the first test piece is lighter than that of the second test piece, it is determined that the first test piece is more subject to rusting than the second test piece. It is possible to realize “The first metal material is less subject to rusting than the second metal material” by using the material used for the first test piece for the protection outer cylinder member and using the material used for the second test piece for the outer cylinder member.

Stainless steels may preferably be used for the outer cylinder member and the protection outer cylinder member for the purpose of protecting the sensor from being damaged by a wayword rock or the like. In this case, by using a stainless steel having a larger Cr content (wt %) than a Cr content (wt %) of the protection outer cylinder for the outer cylinder member, it is possible to use the material less subject to rusting than the material of the protection outer cylinder for the outer cylinder.

In the case of using the stainless steels for the outer cylinder member and the protection outer cylinder member, it is further preferable to keep a content (wt %) of Ni contained in the stainless steel of the outer cylinder member (the first metal material) larger than a content of Ni (wt %) contained in the stainless steel of the protection outer cylinder member (the second metal material)in addition to keeping the content (wt %) of Cr contained in the stainless steel of the outer cylinder member (the first metal material)larger than the content (wt %) of Cr contained in the stainless steel of the protection outer cylinder member (the second metal material).

As a specific material, it is preferable to use SUS310S as the first metal material (for the outer cylinder member) and to use SUS304L as the second metal material (for the protection outer cylinder member). SUS310S is formed of 0.08% or less of C, 1.50% or less of Si, 2.00% or less of Mn, 0.045% or less of P, 0.03% or less of S, 19.00% to 22.00% of Ni, 24.00% to 26.00% of Cr, and residual Fe. Also, SUS304L is formed of 0.03% or less of C, 1.00% or less of Si, 2.00% or less of Mn, 0.045% or less of P, 0.03% or less of S, 9.00% to 13.00% of Ni, 18.00% to 20.00% of Cr, and residual Fe.

Although the invention has been described above in relation to exemplary embodiment thereof, it will be understood by those skilled in the art that variations and modifications can be effected in these exemplary embodiments without departing from the scope and spirit of the invention.

What is claimed is:

1. A sensor comprising:
a sensor element extending in an axial direction, having a periphery and comprising a leading end portion exposed to a gas measurement object;
a metal shell covering the periphery of the sensor element and comprising a rear end side;
an outer cylinder member fixed to the rear end side of the metal shell, the outer cylinder member being composed of a first metal material, having an outer surface and comprising a leading end side; and
a protection outer cylinder member covering the outer surface of the outer cylinder member, the protection outer cylinder being composed of a second metal material and having a circumference;
wherein the outer cylinder member further comprises a sealing part in contact with the circumference of the protection outer cylinder member, and a spaced part positioned closer to the leading end side than the sealing part, opposed to the protection outer cylinder member, and spaced from the protection outer cylinder member; and
wherein the first metal material is less subject to rusting than the second metal material.

2. The sensor according to claim 1, wherein the first metal material and the second metal material are stainless steels, and the first metal material has a larger Cr content (wt %) than the second metal material.

3. The sensor according to claim 2, wherein the first metal material has a larger Ni content (wt %) than the second metal material.

4. The sensor according to claim 3, wherein the first metal material is SUS310S, and the second metal material is SUS304L.

5. The sensor according to claim 1, wherein the sealing part is in contact with the protection outer cylinder member directly.

6. The sensor according to claim 1, wherein the sealing part is in contact with the protection outer cylinder member indirectly via another member.
